(19) United States
(12) Patent Application Publication
(10) Pub. No.: US 2025/0235659 A1
(43) Pub. Date: Jul. 24, 2025

(54) MOTION SICKNESS SUPPRESSING APPARATUS AND MOTION SICKNESS SUPPRESSING METHOD

(71) Applicant: Mitsubishi Electric Corporation, Tokyo (JP)

(72) Inventors: Taichi KOJIMA, Tokyo (JP); Junji HORI, Tokyo (JP); Shintaro WATANABE, Tokyo (JP)

(73) Assignee: Mitsubishi Electric Corporation, Tokyo (JP)

(21) Appl. No.: 18/848,209

(22) PCT Filed: Mar. 29, 2022
(86) PCT No.: PCT/JP2022/015241

§ 371 Date: Sep. 18, 2024

(51) Int. Cl. A61M 21/00

(52) U.S. Cl. CPC A61M 21/00; A61M 2021/005; A61M 2205/3303; A61M 2205/3327

(57) ABSTRACT

A motion sickness suppressing apparatus includes: an acceleration information acquiring unit to acquire information about an acceleration of a vehicle; a gaze position detecting unit to detect a gaze position at which an occupant of the vehicle is gazing; and a video generating unit to generate a video on the basis of the information about the acceleration of the vehicle, and the gaze position.

### TECHNICAL FIELD

The present disclosure relates to a motion sickness suppressing apparatus and a motion sickness suppressing method.

### BACKGROUND ART

The sensory conflict theory is known as a theory that explains the mechanism of occurrence of motion sickness. According to the sensory conflict theory, a sensory mismatch that occurs when the vestibular sensation, visual sensation, and somatic sensation are integrated in a brain causes motion sickness.

There is a conventionally known kinetosis suppressing apparatus that is disclosed to correct a mismatch between the sense of balance and visual sensation of an occupant of a car, and suppress kinetosis (see Patent Literature 1). The apparatus described in Patent Literature 1 corrects the mismatch between the sense of balance and visual sensation of the occupant, and eases kinetosis by detecting an inclination of the head of the occupant caused by a centrifugal force, and displaying a sickness decreasing image that allows the occupant to recognize the inclination of her/his head on the basis of the detected inclination of the head.

### CITATION LIST

### Patent Literatures

Patent Literature 1: JP 2020-131921 A

### SUMMARY OF INVENTION

### Technical Problem

The apparatus described in Patent Literature 1 has a problem that, in some cases, it is not possible to suppress kinetosis, that is, motion sickness, if the occupant in a vehicle such as a car resists movement of the body caused by the influence of an inertial force in a situation where the inertial force is being applied to the occupant. For example, in the situation described above, if the occupant maintains her/his posture resisting the influence of the inertial force to prevent her/his head from being inclined, the apparatus described in Patent Literature 1 cannot prevent motion sickness, because a difference is generated between information from the visual sensation of the occupant and information from the sense of balance, that is, the vestibular sensation, of the occupant.

The present disclosure has been made to solve the problems described above, and an object thereof is to provide a motion sickness suppressing apparatus and a motion sickness suppressing method that make it possible to suppress motion sickness even if an occupant of a vehicle resists movement of her/his body caused by the influence of an inertial force in a situation where the vehicle is accelerating.

### Solution to Problem

A motion sickness suppressing apparatus according to the present disclosure includes: an acceleration information acquiring unit to acquire information about an acceleration of a vehicle; a gaze position detecting unit to detect a gaze position at which an occupant of the vehicle is gazing; and a video generating unit to generate a video on a basis of the information about the acceleration of the vehicle, and the gaze position.

### Advantageous Effects of Invention

The present disclosure makes it possible to suppress motion sickness even if an occupant of a vehicle resists a movement of her/his body caused by the influence of an inertial force in a situation where the vehicle is accelerating.

### BRIEF DESCRIPTION OF DRAWINGS

FIG. 1 is a block diagram depicting the schematic configuration of a motion sickness suppressing apparatus according to a first embodiment.

FIG. 2 is a figure depicting a display example of a suppressing video according to the first embodiment.

FIG. 3A is a figure depicting an example in which one linear suppressing video is displayed according to the first embodiment, FIG. 3B is a figure depicting an example in which two linear suppressing videos are displayed according to the first embodiment, FIG. 3C is a figure depicting an example in which two linear suppressing videos are displayed according to the first embodiment, and FIG. 3D is a figure depicting an example in which two circular suppressing videos are displayed according to the first embodiment.

FIG. 4 is a flowchart depicting a process performed by the motion sickness suppressing apparatus according to the first embodiment.

FIG. 5 is a block diagram depicting the schematic configuration of a motion sickness suppressing apparatus according to a second embodiment.

FIG. 6 is a flowchart depicting a process performed by the motion sickness suppressing apparatus according to the second embodiment.

FIG. 7 is a block diagram depicting the schematic configuration of a motion sickness suppressing apparatus according to a third embodiment.

FIG. 8A is a figure depicting an example in which a suppressing video is displayed on a steering wheel, according to the third embodiment, and FIG. 8A is a figure depicting an example in which a suppressing video is displayed on a door, according to the third embodiment.

FIG. 9 is a flowchart depicting a process performed by the motion sickness suppressing apparatus according to the third embodiment.

FIG. 10 is a block diagram depicting an example of the hardware configuration of the motion sickness suppressing apparatuses according to the first to third embodiments.

FIG. 11 is a block diagram depicting an example of the hardware configuration of the motion sickness suppressing apparatuses according to the first to third embodiments.

### DESCRIPTION OF EMBODIMENTS

Hereinbelow, embodiments according to the present disclosure are explained in detail with reference to the figures.

### First Embodiment

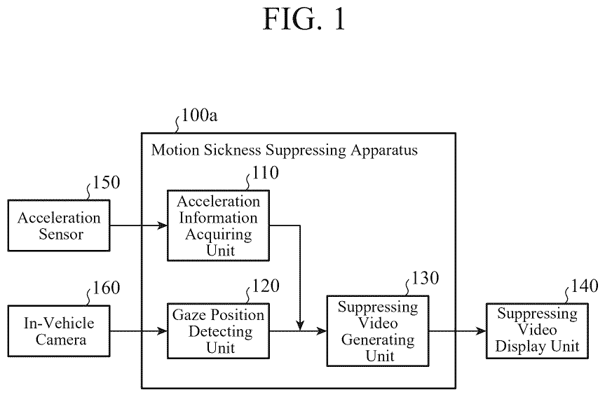

FIG. 1 is a block diagram depicting the schematic configuration of a motion sickness suppressing apparatus 100a according to a first embodiment. The motion sickness suppressing apparatus 100a executes a motion sickness suppressing method for suppressing motion sickness of an occupant of a car which is a vehicle according to the first embodiment. As depicted in FIG. 1, the motion sickness suppressing apparatus 100a includes an acceleration information acquiring unit 110, a gaze position detecting unit 120, and a suppressing video generating unit 130. Note that, in the following explanation, a forward-facing direction which a driver seated in the driver's seat of a car, which is not depicted, faces is defined as the front direction, and, on the basis of this definition, the front, rear, left, and right directions are defined.

The acceleration information acquiring unit 110 acquires information about an acceleration of the car from an acceleration sensor 150 installed on the car. Note that, in the first embodiment, the information about the acceleration of the car is information correlated in some way with the acceleration of the car, and includes information computed on the basis of the acceleration of the car. For example, the acceleration information acquiring unit 110 acquires a measurement value of the acceleration of the car from the acceleration sensor 150, and computes and acquires, on the basis of the measurement value acquired from the acceleration sensor, the direction of the resultant force of a gravitational force applied to the occupant of the car (hereinafter, also referred to as the “occupant” simply) and an inertial force applied to the occupant, and the magnitude of the inertial force applied to the occupant.

For example, on the basis of the measurement value acquired from the acceleration sensor, the acceleration information acquiring unit 110 computes the direction of the gravitational force applied to the occupant, the direction of the inertial force applied to the occupant, and the magnitude of the inertial force applied to the occupant. In addition, on the basis of the direction of the gravitational force applied to the occupant, the direction of the inertial force applied to the occupant, and the magnitude of the inertial force applied to the occupant, the acceleration information acquiring unit 110 computes the direction of the resultant force (hereinafter, also referred to as the “resultant force direction”) of the gravitational force applied to the occupant and the inertial force applied to the occupant.

In addition, for example, the acceleration information acquiring unit 110 processes the measurement value of the acceleration sensor 150 using a low pass filter in order to remove, from the measurement value of the acceleration sensor 150, high-frequency noise generated depending on road surface conditions such as small irregularities of a road surface. The acceleration information acquiring unit 110 outputs, to the suppressing video generating unit 130, the acquired information about the direction of the acceleration applied to the occupant of the car, and the acquired information about the magnitude of the acceleration applied to the occupant of the car.

The gaze position detecting unit 120 detects a gaze position at which the occupant is gazing. For example, on the basis of image information acquired from an in-vehicle camera 160 that captures images of the interior of the car, the gaze position detecting unit 120 detects the gaze position at which the occupant is gazing.

The in-vehicle camera 160 is a device for acquiring the line-of-sight direction of a target occupant by image-capturing. It is sufficient if the in-vehicle camera 160 has image resolution necessary for acquiring the line-of-sight direction of the occupant, and the image information to be acquired may be a grayscale image, a Red Green Blue (RGB) image, or an infrared (IR) image. The in-vehicle camera 160 is, for example, a Charge Coupled Device (CCD), and is disposed at a position where the in-vehicle camera 160 can capture images of the eyeballs of the target occupant. The in-vehicle camera 160 is, for example, disposed in such a manner that the in-vehicle camera 160 captures images of the target occupant from a position at which the in-vehicle camera 160 directly faces the front face of the body of the occupant in a case where the occupant has the front face of her/his body facing the advancing direction of the car. In addition, the in-vehicle camera 160 may be disposed at a middle portion of the car such as the center console of the car, and capture images of the eyeballs of a plurality of occupants in the car.

In addition, for example, the gaze position detecting unit 120 detects the line-of-sight direction of the occupant on the basis of the image information acquired from the in-vehicle camera 160, and detects the gaze position on the basis of the line-of-sight direction of the occupant and changes of the line-of-sight direction. For example, the gaze position detecting unit 120 detects the line-of-sight direction by the corneal reflex method. The corneal reflex method is an approach to measuring eyeball movements on the basis of the position of a corneal reflex image that appears brightly when a cornea is irradiated with light emitted from a point light source.

In addition, the gaze position detecting unit 120 may be configured to detect the line-of-sight direction of the occupant by electrooculography, the search coil method, or the scleral reflex method. Electrooculography is an approach that pays attention to the fact that voltage changes of an eyeball have an almost proportional relationship with the rotation angle of the eyeball. Skin electrodes are attached around an eye, and eyeball movements are measured from voltage changes of the eyeball. The search coil method is an approach in which a coil is attached to the periphery of a contact lens, the wearer of the lens is positioned in a uniform AC magnetic field, and eyeball movements are measured by taking out an induced current proportional to rotation of the eyeball. The scleral reflex method is an approach in which a boundary portion between the iris and the white is irradiated with a weak infrared ray, and eyeball movements are measured by capturing reflection light from the boundary portion with a sensor.

In addition, for example, the gaze position detecting unit 120 detects the gaze position on the basis of the line-of-sight direction of the occupant in a predetermined length of time in a case where a change amount of the line-of-sight direction of the occupant in the predetermined length of time is equal to or smaller than a preset threshold. Specifically, the gaze position detecting unit 120 detects, as the gaze position, a position at which an imaginary straight line extending from the position of the eyeballs of the occupant along the average direction of the line-of-sight direction of the occupant in the predetermined length of time crosses the inner surface of the interior of the car, in a case where a change amount of the line-of-sight direction of the occupant in the predetermined length of time is equal to or smaller than the preset threshold. The gaze position detecting unit 120 outputs information about the detected gaze position to the suppressing video generating unit 130. Note that the gaze position detecting unit 120 may be configured to output the information about the detected gaze position to the suppressing video generating unit 130 only in a case where the detected gaze position is positioned in a display area of a suppressing video display unit 140 mentioned later.

The suppressing video generating unit 130 generates a suppressing video for suppressing motion sickness of the occupant on the basis of the information about the acceleration of the car input from the acceleration information acquiring unit 110, and the information about the gaze position input from the gaze position detecting unit 120. For example, the suppressing video generating unit 130 computes a direction orthogonal to the resultant force direction, generates a suppressing video disposed at the gaze position in such a manner that the suppressing video lies along the computed direction, and causes the suppressing video display unit 140 to display the generated suppressing video. The suppressing video generating unit 130 is included in a video generating unit in the first embodiment. In addition, the suppressing video generated by the suppressing video generating unit 130 is included in a video in the first embodiment.

Note that the suppressing video generating unit 130 may be configured not to generate the suppressing video in a case where the magnitude of the inertial force applied to the occupant acquired by the acceleration information acquiring unit 110 is equal to or greater than a preset predetermined threshold. The suppressing video generating unit 130 configured in this manner can prevent the suppressing video from making the occupant feel annoyed or uncomfortable in a case where the acceleration of the car is high, and a sufficient motion sickness suppressing effect of the suppressing video cannot be expected.

The suppressing video display unit 140 displays the generated suppressing video in a display area where the suppressing video display unit 140 can display the video. For example, the suppressing video display unit 140 may be a vehicle-mounted display, a Head-Up Display (HUD), a display apparatus provided on the In-Panel, or the like, or may be a projector that projects the video onto the inner surface of the interior of the car, or the like. Note that, for example, the “HUD” is a display that makes information directly visible in the visual field of a human by projecting images onto a transparent optical glass element. In addition, the “In-Panel” is an abbreviation of an instrument panel, and is a dashboard installed at a front portion of the driver's seat of the car.

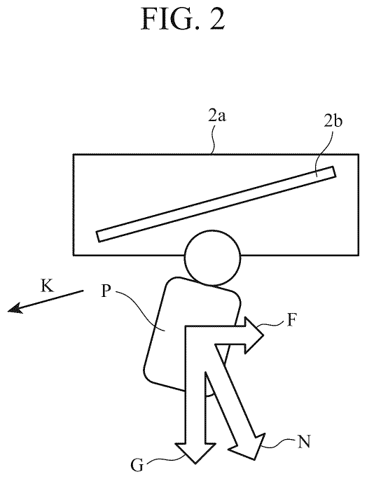

FIG. 2 is a figure depicting a display example of the suppressing video according to the first embodiment. Specifically, FIG. 2 is a figure depicting a display example of the suppressing video in a state where a centrifugal force F as the inertial force is being applied to an occupant P of a car in a case where the car is traveling on a left-hand curve road. For example, in a case where the car is traveling on a left-hand curve road, the acceleration information acquiring unit 110 computes the direction of a resultant force N of the centrifugal force F acting on the occupant P in the right direction and a gravitational force G, that is, calculates the resultant force direction. In addition, in such a case, for example, due to projection of the resultant force direction on a surface parallel to the display surface of a display area 2a of the suppressing video display unit 140, the acceleration information acquiring unit 110 corrects the resultant force direction in such a manner that the resultant force direction lies along the display surface.

In addition, for example, the suppressing video generating unit 130 generates a suppressing video 2b disposed at a position in accordance with the gaze position in such a manner that the suppressing video 2b lies along a direction K orthogonal to the corrected post-correction resultant force direction, and causes the suppressing video 2b to be displayed at a position in accordance with the gaze position of the display area 2a of the suppressing video display unit 140. By defining a reference plane of the car as an imaginary plane that is fixed relative to the car and lying along the front, rear, left, and right directions of the car in a state where the car is disposed on a horizontal plane, the suppressing video 2b in a case where the car is traveling on a left-hand curve road is displayed in such a manner that the suppressing video 2b is inclined counterclockwise relative to the reference plane of the car, when seen in the front direction.

Note that, in a case where an imaginary plane defined by the vector of the gravitational force applied to the occupant and the vector of the inertial force applied to the occupant is parallel to the surface of the display area 2a of the suppressing video display unit 140, the acceleration information acquiring unit 110 need not to correct the resultant force direction.

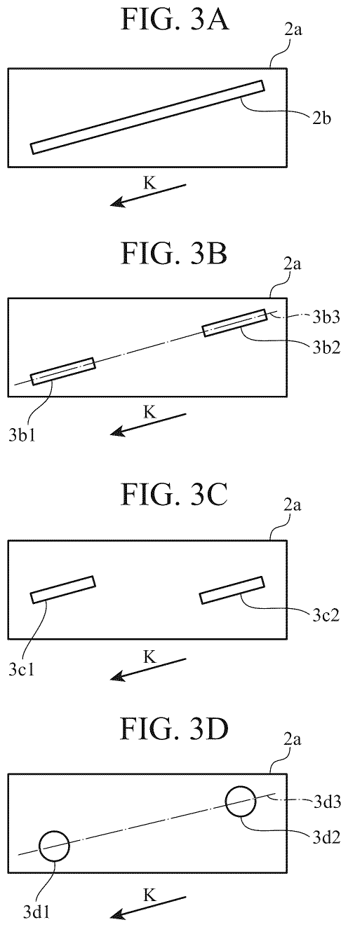

In addition, the suppressing video is not necessarily the one depicted in FIG. 2. FIG. 3A to FIG. 3D are figures depicting display examples of the suppressing video that the suppressing video generating unit 130 causes to be displayed in the display area 2a of the suppressing video display unit 140. FIG. 3A is a figure depicting an example in which one linear suppressing video 2b is displayed according to the first embodiment. FIG. 3B is a figure depicting an example in which two linear suppressing videos 3b1 and 3b2 are displayed according to the first embodiment. The suppressing videos 3b1 and 3b2 are disposed at a certain distance from each other on an imaginary straight line 3b3 disposed along the direction K in such a manner that the suppressing videos 3b1 and 3b2 lie along the imaginary straight line 3b3. The suppressing videos 3b1 and 3b2 may be disposed in such a manner that the gaze position is positioned between the suppressing video 3b1 and the suppressing video 3b2.

FIG. 3C is a figure depicting an example in which two linear suppressing videos 3cl and 3c2 are displayed according to the first embodiment. For example, the suppressing videos 3cl and 3c2 are disposed at distances from each other in the direction K and a direction crossing the direction K. In addition, for example, the suppressing videos 3cl and 3c2 are disposed in such a manner that their vertical positions are the same. The suppressing videos 3cl and 3c2 may be disposed in such a manner that the gaze position is positioned between the suppressing video 3cl and the suppressing video 3c2.

FIG. 3D is a figure depicting an example in which two circular suppressing videos are displayed according to the first embodiment. For example, suppressing videos 3d1 and 3d2 are disposed at a certain distance from each other on an imaginary straight line 3d3 disposed along the direction K. The suppressing videos 3d1 and 3d2 may be disposed in such a manner that the gaze position is positioned between the suppressing video 3d1 and the suppressing video 3d2. Note that the shapes, positions, and display modes of suppressing videos are not necessarily those mentioned above. It is sufficient if the suppressing videos can be visually displayed to inform that the body of the occupant is inclined in a direction opposite to a direction in which the body of the occupant is supposed to incline due to the inertial force relative to the reference plane of the car, and it is possible that a variety of videos are used as the suppressing videos. For example, suppressing videos may not have uniform transparency, colors, contrast, or the like, and may include combinations of videos with various sizes and forms. In addition, for example, the suppressing videos 2b, 3b1, 3b2, 3c1, 3c2, 3d1, and 3d2 may be displayed in such a manner that the suppressing videos 2b, 3b1, 3b2, 3cl, 3c2, 3d1, and 3d2 are arranged in the peripheral visual field of the occupant. Details of this are mentioned later.

In addition, for example, suppressing videos may be videos for displaying some information, may be videos for displaying a travel route of the car, may be videos for displaying audio, movie, advertisement, or other video content, or may be videos for displaying information about the car such as the speed of the car or the traveled distance of the car.

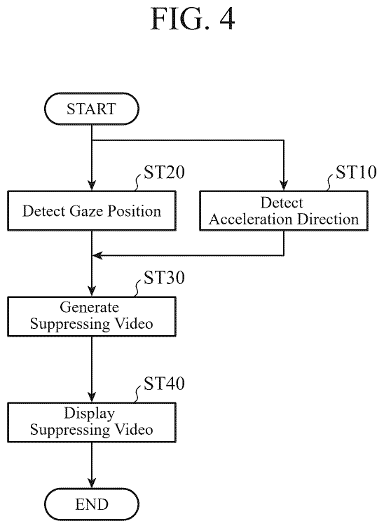

Next, a process performed by the motion sickness suppressing apparatus 100a is explained with reference to FIG. 4. FIG. 4 is a flowchart depicting the process performed by the motion sickness suppressing apparatus 100a according to the first embodiment. The process performed by the motion sickness suppressing apparatus 100a depicted in FIG. 4 has: Step ST10 at which the acceleration information acquiring unit 110 acquires the information about the acceleration of the car; Step ST20 at which the gaze position detecting unit 120 detects the gaze position at which the occupant is gazing; Step ST30 at which the suppressing video generating unit 130 generates the suppressing video on the basis of the information about the acceleration of the car, and the gaze position; and Step ST40 at which the suppressing video generating unit 130 causes the suppressing video display unit 140 to display the suppressing video.

At Step ST10, for example, the motion sickness suppressing apparatus 100a acquires the information about the acceleration of the car on the basis of the information from the acceleration sensor 150.

At Step ST20, for example, the motion sickness suppressing apparatus 100a detects the gaze position of the occupant on the basis of the line-of-sight direction of the occupant.

At Step ST30, for example, the motion sickness suppressing apparatus 100a generates the suppressing video disposed along the direction orthogonal to the resultant force direction on the basis of the gaze position on the display area 2a of the suppressing video display unit 140.

At Step ST40, the motion sickness suppressing apparatus 100a causes the suppressing video display unit 140 to display the generated suppressing video.

Since, as explained thus far, in the motion sickness suppressing apparatus 100a according to the first embodiment, the suppressing video generating unit 130 generates the suppressing video on the basis of the information about the acceleration of the car, and the gaze position, it becomes possible to suppress motion sickness even if the occupant of the car resists movement of her/his body caused by the influence of an inertial force in a situation where the car is accelerating.

Note that whereas, in the first embodiment, the acceleration information acquiring unit 110 is configured to acquire the information about the acceleration of the car from the acceleration sensor 150, and the acceleration information acquiring unit 110 is configured to compute the direction of the resultant force of the gravitational force applied to the occupant and the inertial force applied to the occupant, and the magnitude of the inertial force applied to the occupant on the basis of the information acquired from the acceleration sensor 150, this is not the sole example. It is sufficient if the acceleration information acquiring unit acquires at least one of: information about the direction of the gravitational force applied to the car (occupant); information about the direction of the resultant force of the gravitational force applied to the occupant and the inertial force applied to the occupant; and information about the magnitude of the inertial force applied to the occupant. For example, the acceleration information acquiring unit may be configured to compute only the direction of the gravitational force applied to the car on the basis of the acceleration of the car detected by the acceleration sensor, may be configured to compute only the information about the direction of the resultant force of the gravitational force applied to the occupant and the inertial force applied to the occupant, or may be configured to compute only the information about the magnitude of the inertial force applied to the occupant.

In addition, for example, the acceleration information acquiring unit may be configured to acquire information about a horizontal direction computed by the acceleration sensor on the basis of the information about the acceleration of the car, and, on the basis of the information about the horizontal direction, compute the direction of the gravitational force applied to the car. In addition, the acceleration information acquiring unit may be configured to acquire, from the acceleration sensor 150: the information about the direction of the gravitational force applied to the car computed on the basis of the information about the acceleration of the car detected by the acceleration sensor; the information about the direction of the inertial force applied to the occupant; and the information about the magnitude of the inertial force applied to the occupant. In addition, for example, the acceleration information acquiring unit does not necessarily acquire information from one acceleration sensor, but may acquire information from a plurality of acceleration sensors.

In addition, the information about the acceleration of the car acquired by the acceleration information acquiring unit is not necessarily an actual measurement value of the acceleration of the car. The information about the acceleration of the car acquired by the acceleration information acquiring unit may be an estimated value of the acceleration of the car. For example, the information about the acceleration of the car may be an estimated value computed on the basis of the speed of the car and the rotation radius of the car, or may be an estimated value computed on the basis of the inclination of the car. The acceleration information acquiring unit may compute the rotation radius of the car on the basis of the steering angle of the steering wheel, or may compute the rotation radius of the car on the basis of positional information about the car acquired from a positional information acquiring unit (not depicted), and a travel route of the car predicted from map information. In addition, the acceleration information acquiring unit may compute the inclination of the car on the basis of image information from a vehicle outside camera (not depicted) that captures images of the outside of the car.

In addition, whereas the suppressing video generating unit 130 is configured to generate the suppressing video on the basis of the direction of the resultant force of the gravitational force applied to the occupant and the inertial force applied to the occupant in the first embodiment, this is not the sole example. It is sufficient if the suppressing video generating unit is configured to generate the video on the basis of the information about the acceleration of the vehicle acquired by the acceleration information acquiring unit, and the gaze position detected by the gaze position detecting unit. For example, the suppressing video generating unit may be configured to generate the suppressing video on the basis of the direction of the gravitational force applied to the car acquired by the acceleration information acquiring unit, and the gaze position. Specifically, the suppressing video generating unit may be configured to generate the suppressing video to be disposed along a direction orthogonal to the direction of the gravitational force applied to the car, or may be configured to generate the suppressing video to be disposed along a direction at a greater angle to the reference plane of the car than an angle to the direction orthogonal to the direction of the gravitational force applied to the car.

In addition, for example, the suppressing video generating unit may be configured to generate the suppressing video on the basis of the information about the magnitude of the inertial force applied to the occupant acquired by the acceleration information acquiring unit. Specifically, the suppressing video generating unit may be configured to generate the suppressing video to be disposed along a direction at an angle to the reference plane of the car that increases as the magnitude of the inertial force applied to the occupant increases.

### Second Embodiment

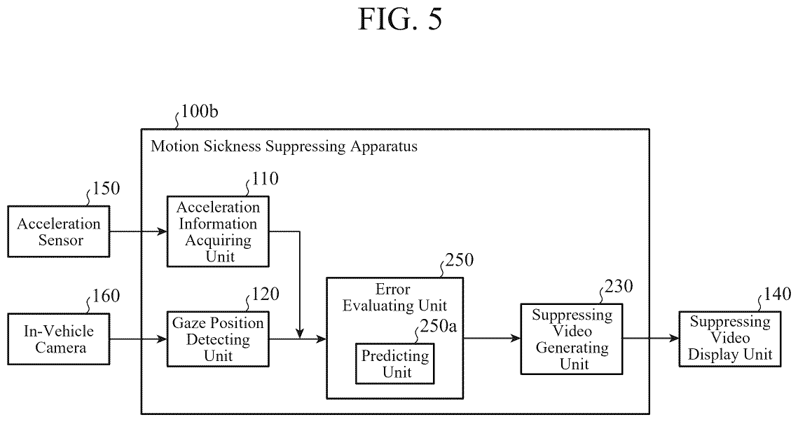

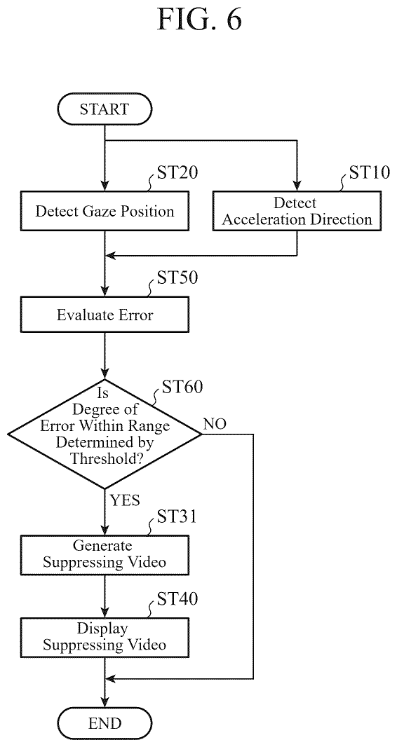

Next, a second embodiment is explained with reference to FIGS. 5 and 6. A motion sickness suppressing apparatus 100b according to the second embodiment is different from the motion sickness suppressing apparatus 100a according to the first embodiment in terms of an error evaluating unit 250 and a suppressing video generating unit 230, but is similar to the motion sickness suppressing apparatus 100a in terms of other configuration and processes. The similar configuration and processes are given identical reference signs, and overlapping explanations are omitted.

FIG. 5 is a block diagram depicting the schematic configuration of the motion sickness suppressing apparatus 100b according to the second embodiment. The motion sickness suppressing apparatus 100b includes an acceleration information acquiring unit 110, a gaze position detecting unit 120, the error evaluating unit 250, and the suppressing video generating unit 230. The acceleration information acquiring unit 110, the gaze position detecting unit 120, and a suppressing video display unit 140 of the motion sickness suppressing apparatus 100b according to the second embodiment are similar to the acceleration information acquiring unit 110, the gaze position detecting unit 120, and the suppressing video display unit 140 of the motion sickness suppressing apparatus 100a according to the first embodiment.

The error evaluating unit 250 evaluates a mismatch, that is, the degree of an error, between the vestibular sensation and visual sensation of an occupant on the basis of information output by the acceleration information acquiring unit 110, and information output by the gaze position detecting unit 120. For example, in a case where the acceleration information acquiring unit 110 has acquired an inertial force applied to the occupant, it is predicted that the error of the sense of the occupant increases as the inertial force applied to the occupant increases, and accordingly the error evaluating unit 250 increases the evaluated value of the error as the inertial force applied to the occupant increases, that is, as the acceleration of a car increases. In other words, the error evaluating unit 250 computes the value of the error depending on the magnitude of the acceleration of the car.

In addition, the error evaluating unit 250 has a predicting unit 250a. The predicting unit 250a predicts the acceleration of the car. For example, on the basis of image information from a vehicle outside camera (not depicted) that captures images of the outside of the car, the predicting unit 250a acquires information about a road such as the inclination of the road surface, irregularities of the road surface, objects on the road, or a travel route, and predicts the acceleration of the car on the basis of the information about the road. In addition, for example, the predicting unit 250a predicts the acceleration of the car on the basis of positional information about the car acquired from a positional information acquiring unit (not depicted), and a travel route of the car predicted from map information. Note that the predicting unit 250a is included in an acceleration predicting unit in the first embodiment. The error evaluating unit 250 outputs, to the suppressing video generating unit 230, a result of the evaluation of the error, a result of the prediction of the acceleration of the car, the information from the acceleration information acquiring unit 110, and the information from the gaze position detecting unit 120. Note that the error evaluating unit 250 may be configured to evaluate the error by using a model having human-like acceleration sensibility. It is known that typically the ease of perception of the acceleration by the occupant changes depending on the direction and frequency of acceleration. Because of this, for example, the error evaluating unit 250 may increase the evaluated value of the error in a case where the acceleration of a force applied to the occupant has a direction and frequency that can be perceived easily.

The suppressing video generating unit 230 generates a suppressing video on the basis of the information input from the error evaluating unit 250. For example, the suppressing video generating unit 230 generates the suppressing video according to the result of the evaluation of the error by the error evaluating unit 250. In other words, the suppressing video generating unit 230 generates the suppressing video depending on the magnitude of the acceleration of the car. For example, the suppressing video generating unit 230 generates the suppressing video in such a manner that the level of awareness of the occupant to be increased by the suppressing video increases as the error represented by the evaluation result of the error evaluating unit 250 increases. For example, the suppressing video generating unit 230 generates the suppressing video with an inclination relative to a reference plane of the car that increases as the error represented by the evaluation result of the error evaluating unit 250 increases. In addition, for example, the suppressing video generating unit 230 generates the suppressing video with a size and contrast that increase as the error represented by the evaluation result of the error evaluating unit 250, in such a manner that the occupant can perceive the suppressing video easily.

Note that, in a case where the error represented by the evaluation result of the error evaluating unit 250 is greater than a predetermined error, there is a possibility that the occupant feels uncomfortable due to the suppressing video being displayed. Because of this, the suppressing video generating unit 230 may be configured not to generate the suppressing video in a case where the error represented by the evaluation result of the error evaluating unit 250 is equal to or greater than a preset threshold, in other words, in a case where the magnitude of the acceleration of the car is equal to or greater than the preset threshold. In addition, in such a case, the suppressing video generating unit 230 may cause the suppressing video display unit 140 to display information prompting the occupant to suppress motion sickness by another method other than viewing the suppressing video such as: information prompting the occupant to stop viewing and listening to media content; information prompting the occupant to close her/his eyes, and relax; or information prompting the occupant to stop the car at a safe location.

In addition, for example, the suppressing video generating unit 230 generates the suppressing video on the basis of the acceleration of the car predicted by the predicting unit 250a. Specifically, on the basis of the acceleration of the car predicted by the predicting unit 250a, the suppressing video generating unit 230 generates the suppressing video according to an acceleration at which the vehicle will be traveling a predetermined length of time after, for example several seconds after. By displaying the suppressing video generated on the basis of the acceleration of the car predicted by the predicting unit 250a in this manner, before the timing at which an inertial force is actually applied to her/him, the occupant can know the inertial force that is to be applied to her/him, and accordingly it becomes easier for the occupant to move her/his body to resist movement of the body caused by the influence of the inertial force.

Next, a process performed by the motion sickness suppressing apparatus 100b is explained with reference to FIG. 6. FIG. 6 is a flowchart depicting the process performed by the motion sickness suppressing apparatus 100b according to the second embodiment. The process performed by the motion sickness suppressing apparatus 100b depicted in FIG. 6 has: Step ST10 at which the acceleration information acquiring unit 110 acquires the information about the acceleration of the car; Step ST20 at which the gaze position detecting unit 120 detects the gaze position at which the occupant is gazing; Step ST50 at which the error evaluating unit 250 evaluates the degree of the error as determined by the difference between the vestibular sensation and visual sensation of the occupant; Step ST60 at which the suppressing video generating unit 230 assesses whether or not the error represented by an evaluation result of the error evaluating unit 250 is smaller than the threshold; Step ST31 at which the suppressing video generating unit 230 generates the suppressing video on the basis of the information from the error evaluating unit 250; and Step ST40 at which the suppressing video generating unit 230 causes the suppressing video display unit 140 to display the suppressing video.

Step ST10, Step ST20, and Step ST40 performed by the motion sickness suppressing apparatus 100b according to the second embodiment are similar to Step ST10, Step ST20, and Step ST40 performed by the motion sickness suppressing apparatus 100a according to the first embodiment, respectively.

At Step ST50, for example, the motion sickness suppressing apparatus 100b evaluates the estimated value of the error of the sense of the occupant depending on the magnitude of the acceleration of the car.

At Step ST60, the motion sickness suppressing apparatus 100b assesses whether or not the error represented by a result of the evaluation at Step ST50 is smaller than the threshold, and, in a case where the error is smaller than the threshold (YES at Step ST60), Step ST31 is performed. In a case where the error is equal to or greater than the threshold (NO at Step ST60), the motion sickness suppressing apparatus 100b ends the process.

At Step ST31, for example, the motion sickness suppressing apparatus 100b generates the suppressing video on the basis of the result of the evaluation of the error at Step ST50, the resultant force direction, and the gaze position.

Since, as explained thus far, the motion sickness suppressing apparatus 100b according to the second embodiment generates the suppressing video depending on the magnitude of the acceleration of the car, for example, it becomes possible to make a proposal to the occupant to suppress motion sickness by another method instead of displaying the suppressing image in a case where the magnitude of the acceleration of the car is equal to or greater than the preset threshold.

### Third Embodiment

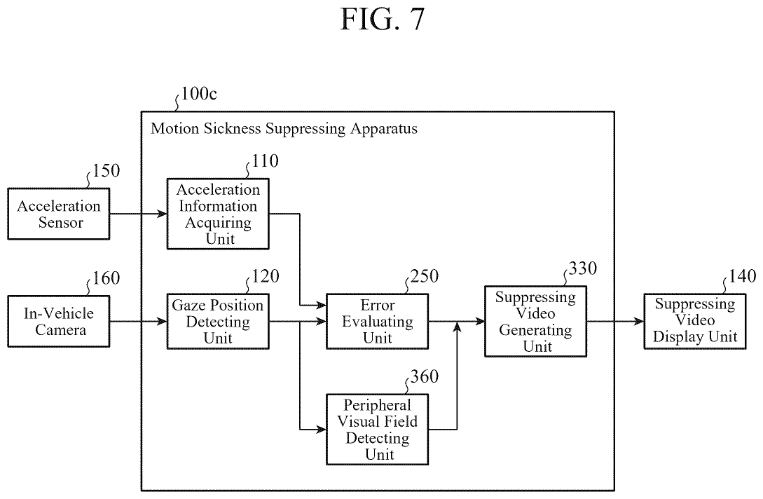

Next, a third embodiment is explained with reference to FIGS. 7 to 9. A motion sickness suppressing apparatus 100c according to the third embodiment is different from the motion sickness suppressing apparatus 100b according to the second embodiment in terms of a peripheral visual field detecting unit 360 and a suppressing video generating unit 330, but is similar to the motion sickness suppressing apparatus 100b in terms of other configuration and processes. The similar configuration and processes are given identical reference signs, and overlapping explanations are omitted.

A block diagram depicting the schematic configuration of the motion sickness suppressing apparatus 100c according to the third embodiment is shown. The motion sickness suppressing apparatus 100c includes an acceleration information acquiring unit 110, a gaze position detecting unit 120, an error evaluating unit 250, the peripheral visual field detecting unit 360, and the suppressing video generating unit 330. The acceleration information acquiring unit 110, the gaze position detecting unit 120, the error evaluating unit 250, and a suppressing video display unit 140 of the motion sickness suppressing apparatus 100c according to the third embodiment are similar to the acceleration information acquiring unit 110, the gaze position detecting unit 120, the error evaluating unit 250, and the suppressing video display unit 140 of the motion sickness suppressing apparatus 100b according to the second embodiment, respectively.

The peripheral visual field detecting unit 360 detects a peripheral visual field from information about a gaze position received from the gaze position detecting unit 120. For example, the peripheral visual field detecting unit 360 detects, on the display surface of a display area 2a and as the peripheral visual field, an area outside a first area including the gaze position and inside a second area including the first area. In addition, for example, the peripheral visual field detecting unit 360 detects, on the display surface of the display area 2a and as the peripheral visual field, an area that is centered on the gaze position, outside a circle having a first radius, and inside a circle including the circle and having a second radius greater than the first radius. In addition, for example, the peripheral visual field detecting unit 360 detects, as the peripheral visual field, an area outside a conical area with a predetermined apex angle having, as the centerline, an imaginary straight line that coincides with the line-of-sight direction of the occupant, and inside a predetermined area, for example the visual field of the occupant, including the area. Specifically, the peripheral visual field detecting unit 360 detects, as the peripheral visual field, an area outside a conical area with an apex angle of 60° (with a half apex angle of 30°) having, as the centerline, the imaginary straight line that coincides with the line-of-sight direction of the occupant, and inside the predetermined area including the area. The peripheral visual field detecting unit 360 outputs information about the detected area of the peripheral visual field to the suppressing video generating unit 330.

The peripheral visual field means an area inside the viewing range of the human and outside the central visual field, and, typically, the angle of the central visual field is approximately 30° from the imaginary straight line that coincides with the line-of-sight direction. In addition, it is known that, typically, the visual sensation in the peripheral visual field has lower image resolution than that of the visual sensation in the central visual field, but is more sensitive to motions than the visual sensation in the central visual field is. For example, the motion sickness suppressing apparatus 100c according to the third embodiment does not cause a suppressing video to be displayed in the central visual field of the occupant, but causes the suppressing video to be displayed in the peripheral visual field of the occupant. Thereby, it is made possible to make it unlikely for the occupant to feel annoyed as compared to a case where the suppressing video is displayed at the gaze position, and it is made possible to cause the occupant to more intensely recognize a motion of the suppressing video, and to suppress motion sickness effectively.

The suppressing video generating unit 330 generates the suppressing video on the basis of information from the error evaluating unit 250, and information from the peripheral visual field detecting unit 360. For example, on the basis of a result of the detection of the peripheral visual field by the peripheral visual field detecting unit 360, the suppressing video generating unit 330 generates the suppressing video based on a result of evaluation of an error by the error evaluating unit 250 in an area which is the peripheral visual field of the occupant on the display surface of the display area 2a, and causes the suppressing video display unit 140 to display the suppressing video.

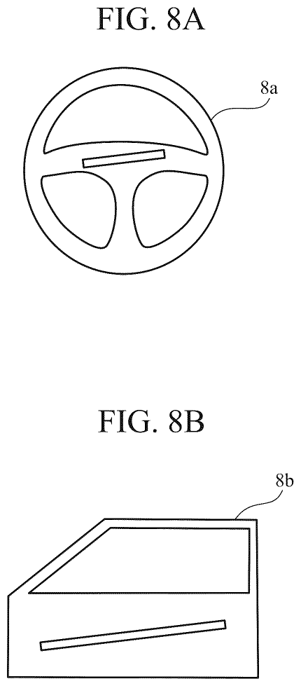

FIG. 8A is a figure depicting an example in which the suppressing video is displayed on a steering wheel 8a, according to the third embodiment, and FIG. 8A is a figure depicting an example in which the suppressing video is displayed on a door 8b, according to the third embodiment. In this manner, the suppressing video generating unit 330 may cause the suppressing video to be displayed at a location where the display area 2a of the suppressing video display unit 140 can be formed such as a surface of the steering wheel 8a which is in the peripheral visual field of the occupant, or a surface of the door 8b (the inner surface of the interior of the car) which is in the peripheral visual field of the occupant.

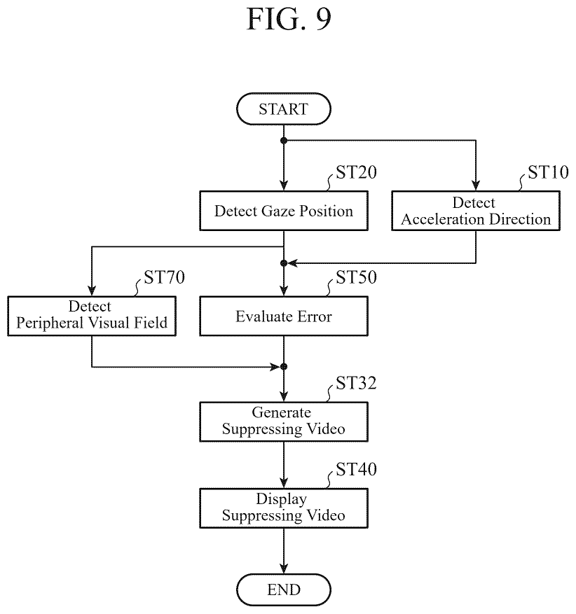

Next, a process performed by the motion sickness suppressing apparatus 100c is explained with reference to FIG. 9. FIG. 9 is a flowchart depicting the process performed by the motion sickness suppressing apparatus 100c according to the third embodiment. The process performed by a motion sickness suppressing apparatus 100a depicted in FIG. 9 has: Step ST10 at which the acceleration information acquiring unit 110 acquires information about the acceleration of the car; Step ST20 at which the gaze position detecting unit 120 detects a gaze position at which the occupant is gazing; Step ST50 at which the error evaluating unit 250 evaluates the degree of the error as determined by the difference between the vestibular sensation and visual sensation of the occupant; Step ST70 at which the peripheral visual field detecting unit 360 detects the peripheral visual field; Step ST32 at which the suppressing video generating unit 330 generates the suppressing video on the basis of the information from the error evaluating unit 250, and the information from the peripheral visual field detecting unit 360; and Step ST40 at which a suppressing video generating unit 130 causes the suppressing video display unit 140 to display the suppressing video.

Step ST10, Step ST20, Step ST50, and Step ST40 performed by the motion sickness suppressing apparatus 100c according to the third embodiment are similar to Step ST10, Step ST20, Step ST50, and Step ST40 performed by the motion sickness suppressing apparatus 100b according to the second embodiment, respectively.

At Step ST60, for example, the motion sickness suppressing apparatus 100c detects, as the peripheral visual field, an area in the visual field of the occupant and outside the central visual field on the basis of a result of the detection of the gaze position.

For example, at Step ST32, the suppressing video is generated on the basis of a result of the evaluation of the error, a resultant force direction, and a result of the detection of the peripheral visual field.

As explained thus far, the motion sickness suppressing apparatus 100c according to the third embodiment causes the suppressing video to be displayed in the peripheral visual field of the occupant, and thereby can suppress motion sickness more effectively without causing the occupant to feel annoyed due to the suppressing video.

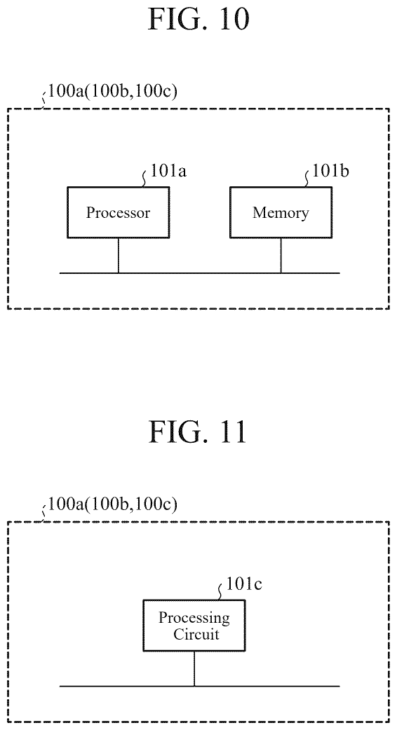

Next, the hardware configuration of the signal processing unit 11S mentioned above is explained with reference to FIG. 10 and FIG. 11. FIG. 10 is a block diagram depicting an example of the hardware configuration of the motion sickness suppressing apparatuses according to the first to third embodiments, and FIG. 4 is a block diagram depicting an example, which is different from that depicted in the FIG. 10, of the hardware configuration of the motion sickness suppressing apparatuses according to the first to third embodiments.

As depicted in FIG. 10, for example, the motion sickness suppressing apparatus 100a includes at least one processor 101a and a memory 101b. For example, the processor 101a is a Central Processing Unit (CPU) to execute programs stored on the memory 101b. In this case, functions of the motion sickness suppressing apparatus 100a are implemented by software, firmware, or a combination of software and firmware. Software and firmware are stored as programs on the memory 101b. Thereby, programs for implementing functions of the motion sickness suppressing apparatus 100a, for example the motion sickness suppressing method according to the first embodiment, are executed by the processor 101a.

The memory 101b is a computer-readable recording medium, and, for example, is configured by using a volatile memory such as a Random Access Memory (RAM) or a Read Only Memory (ROM), a non-volatile memory, or a combination of a volatile memory and a non-volatile memory.

In addition, for example, as depicted in FIG. 11, the motion sickness suppressing apparatus 100a may be configured by using a processing circuit 101c as dedicated hardware. For example, the processing circuit 101c is configured by using a single circuit, a composite circuit, a programmed processor, a parallel-programmed processor, an Application Specific Integrated Circuit (ASIC), a Field Programmable Gate Array (FPGA), or a combination of these. In this case, functions of the motion sickness suppressing apparatus 100a are implemented by the processing circuit 101c executing programs.

Note that since the hardware configuration of the motion sickness suppressing apparatus 100b according to the second embodiment and the motion sickness suppressing apparatus 100c according to the third embodiment is similar to that of the motion sickness suppressing apparatus 100a according to the first embodiment, explanations thereof are omitted.

In addition, an inertial force applied to an occupant is not necessarily a centrifugal force in any of the embodiments mentioned above. For example, in a case where the occupant is oriented in the right direction or the left direction relative to the advancing direction, a suppressing video according to an inertial force caused by acceleration or deceleration of a car may be displayed on a side surface or the like of the interior of the car, or the resultant force of an inertial force and centrifugal force caused by the acceleration or the deceleration of the car may be treated as an inertial force applied to the occupant, and a suppressing video according to the inertial force may be displayed.

In addition, whereas a vehicle is a car in any of the examples explained in the embodiments mentioned above, this is not the sole example. It is sufficient if the vehicle is a movable object in which an occupant gets in, and, for example, the vehicle may be a ship, an airplane, an automobile, a train, a passenger vehicle, a work vehicle, or the like.

Note that, in the present disclosure, any combinations of embodiments, modifications of any components in embodiments, or omission of any components in embodiments are/is possible.

### INDUSTRIAL APPLICABILITY

A motion sickness suppressing apparatus and a motion sickness suppressing method according to the present disclosure can be used for suppressing motion sickness of an occupant.

### REFERENCE SIGNS LIST

100a, 100b, 100c: motion sickness suppressing apparatus, 110: acceleration information acquiring unit, 120: gaze position detecting unit, 130, 230, 330: suppressing video generating unit (video generating unit), 250a: predicting unit (acceleration predicting unit), 2b, 3b1, 3b2, 3cl, 3c2, 3d1, 3d2: suppressing video (video), F: centrifugal force (inertial force), G: gravitational force, N: resultant force, P: occupant

1. A motion sickness suppressing apparatus comprising processing circuitry
to acquire information about an acceleration of a vehicle,
to detect a gaze position at which an occupant of the vehicle is gazing, and
to generate a suppressing video on a basis of the information about the acceleration of the vehicle, and the gaze position, the suppressing video being arranged along an orthogonal direction of a resultant force of a gravitational force applied to the occupant and an inertial force applied to the occupant, or an orthogonal direction of a gravitational force applied to the vehicle.

2.-9. (canceled)

10. A motion sickness suppressing apparatus comprising processing circuitry
to acquire information about an acceleration of a vehicle,
to detect a gaze position at which an occupant of the vehicle is gazing, and
to generate a suppressing video visually displaying that the occupant is inclined in a direction opposite to a direction in which a body of the occupant is inclined due to an inertial force with respect to a reference plane of the vehicle on a basis of the information about the acceleration of the vehicle and the gaze position.

11. The motion sickness suppressing apparatus according to claim 1, wherein the processing circuitry causes the generated suppressing video to be displayed at a position in accordance with the gaze position.

12. The motion sickness suppressing apparatus according to claim 1, wherein the processing circuitry is further configured to predict the acceleration of the vehicle, and to generate the suppressing video on a basis of the predicted acceleration of the vehicle.

13. The motion sickness suppressing apparatus according to claim 1, wherein the processing circuitry is further configured to generate the suppressing video depending on a magnitude of the acceleration of the vehicle.

14. The motion sickness suppressing apparatus according to claim 1, wherein the processing circuitry is further configured to detect the gaze position on a basis of a line-of-sight direction of the occupant in a predetermined length of time in a case where a change amount of the line-of-sight direction in the predetermined length of time is equal to or smaller than a preset threshold.

15. The motion sickness suppressing apparatus according to claim 1, wherein the processing circuitry is further configured to perform evaluation of a degree of an error between vestibular sensation and visual sensation of the occupant on a basis of the information about the acceleration of the vehicle and the gaze position, and
to generate the suppressing video with a size and contrast that increase as a magnitude of the error represented by a result of the evaluation for making the occupant easily perceive the suppressing video.

16. The motion sickness suppressing apparatus according to claim 1, wherein the processing circuitry causes the suppressing video to be displayed outside a first area including the gaze position and inside a second area including the first area.

17. The motion sickness suppressing apparatus according to claim 10, wherein the processing circuitry causes the generated suppressing video to be displayed at a position in accordance with the gaze position.

18. The motion sickness suppressing apparatus according to claim 10, wherein the processing circuitry is further configured to predict the acceleration of the vehicle, and to generate the suppressing video on a basis of the predicted acceleration of the vehicle.

19. The motion sickness suppressing apparatus according to claim 10, wherein the processing circuitry is further configured to generate the suppressing video depending on a magnitude of the acceleration of the vehicle.

20. The motion sickness suppressing apparatus according to claim 10, wherein the processing circuitry is further configured to detect the gaze position on a basis of a line-of-sight direction of the occupant in a predetermined length of time in a case where a change amount of the line-of-sight direction in the predetermined length of time is equal to or smaller than a preset threshold.

21. The motion sickness suppressing apparatus according to claim 10, wherein the processing circuitry is further configured to perform evaluation of a degree of an error between vestibular sensation and visual sensation of the occupant on a basis of the information about the acceleration of the vehicle and the gaze position, and
to generate the suppressing video with a size and contrast that increase as a magnitude of the error represented by a result of the evaluation for making the occupant easily perceive the suppressing video.

22. The motion sickness suppressing apparatus according to claim 10, wherein the processing circuitry causes the suppressing video to be displayed outside a first area including the gaze position and inside a second area including the first area.

23. A motion sickness suppressing method comprising:
acquiring information about an acceleration of a vehicle;
detecting a gaze position at which an occupant of the vehicle is gazing; and
generating a suppressing video on a basis of the information about the acceleration of the vehicle, and the gaze position, the suppressing video being arranged along an orthogonal direction of a resultant force of a gravitational force applied to the occupant and an inertial force applied to the occupant, or an orthogonal direction of a gravitational force applied to the vehicle.

24. A motion sickness suppressing method comprising:
acquiring information about an acceleration of a vehicle;
detecting a gaze position at which an occupant of the vehicle is gazing; and
generating a suppressing video on a basis of the information about the acceleration of the vehicle, and the gaze position, the suppressing video visually displaying that the occupant is inclined in a direction opposite to a direction in which a body of the occupant is inclined due to an inertial force with respect to a reference plane of the vehicle.
